(19) United States
(12) Patent Application Publication
(10) Pub. No.: US 2020/0237945 A1
(43) Pub. Date: Jul. 30, 2020

(54) Autoclave Biological Indicator Retrieval Device with Steam and Pressure Pass-through Tube

(71) Applicant: Christopher Bradley Goforth, Troy, NC (US)

(72) Inventor: Christopher Bradley Goforth, Troy, NC (US)

(21) Appl. No.: 16/260,035

(22) Filed: Jan. 28, 2019

(51) Int. Cl. A61L 2/28; A61L 2/07

(52) U.S. Cl. CPC A61L 2/28; A61L 2202/122; A61L 2202/123; A61L 2/07

(57) ABSTRACT

During autoclave sterilization, technicians often tighten waste bags too tight not allowing proper steam permeation into the items in the autoclave bag. Additionally, biological indicators (bioindicators) should be used to confirm sterilization of items in the waste bag; however it is recommended that these indicators be placed close to the center of the bag to give a more accurate representation of sterilization. This is not safely feasible due to the need to probe into a waste bag for placement and retrieval of biological indicators after autoclaving. This device addresses both of these technical problems. Placing it in an autoclave bag cinched at the composite collar allows steam to penetrate into the autoclave bag via a pass-through tube close to the center mass of the waste bag. Additionally, this device holds many types of biological indicators allowing them to be safely placed and retrieved from center mass of an autoclave bag.

### CROSS-REFERENCE TO RELATED APPLICATIONS

Not Applicable

### REFERENCE TO SEQUENCE LISTING, ETC.

Not Applicable

### BACKGROUND OF INVENTION

This invention is intended for the medical, laboratory diagnostic and research field. To be used by those using an autoclave for sterilization (hospitals, health clinics, pharmaceutical testing, academia and other microbiological laboratories).

During autoclave sterilization, technicians often tighten waste bags too tight not allowing proper steam permeation into the items in the autoclave bag. Additionally, biological indicators (bioindicators) should be used to confirm sterilization of items in the waste bag; however it is recommended that these indicators be placed close to the center of the bag to give a more accurate representation of sterilization. This is not safely feasible due to the need to probe into a waste bag for placement and retrieval of biological indicators after autoclaving.

### BRIEF SUMMARY OF INVENTION

This device addresses both technical problems. Placing this device in an autoclave bag cinched at the composite collar allows steam to penetrate into the autoclave bag which may allow better sterilization. The upper T-shaped handle protrudes from the autoclave bag when cinched/taped/tied allowing steam to enter. It will exit the copper pass-through tube close to the center mass of the waste bag. Additionally, this device holds various types of biological indicators allowing them to be positioned near the center of the mass in the waste bag. Upon completion of the autoclave sterilization run, the retrieval device is removed easily allowing access to the biological indicator without reaching into a bag of potentially dangerous items. After removal, the autoclave bag can be cinched tight for eventual disposal.

### BRIEF DESCRIPTION OF THE DRAWINGS

FIG. 1. Full view of device.

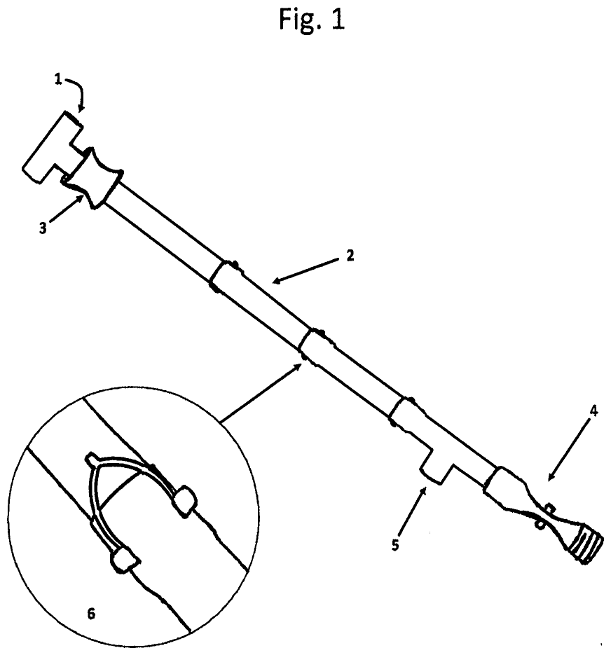

FIG. 2. Detail view of composite end.

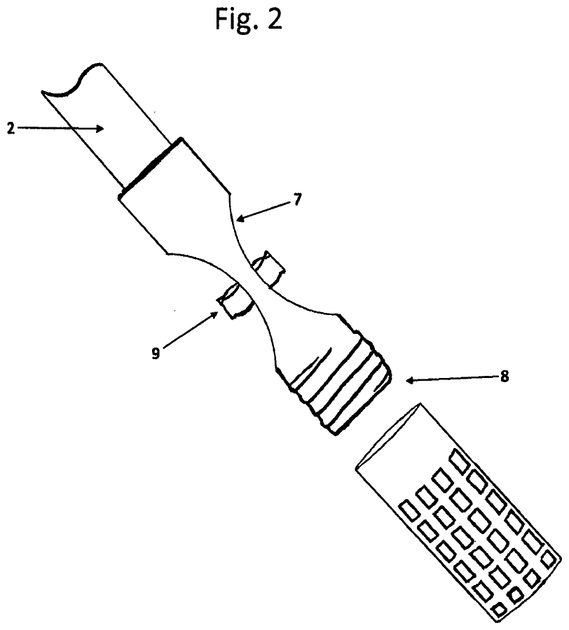

### DETAILED DESCRIPTION OF THE INVENTION

Primary technical features include a copper pass-through tube. This tube has removable sections allowing size adjustment to accommodate various sizes of autoclave bags. A composite plastic biological indicator holder is attached to the base of the pass-through tube. This may prevent excess unintentional heat transfer directly from the copper tube to the biological indicator. This connection of the two dissimilar materials may allow the biological indicator to more accurately report the actual environment in the autoclave bag. The biological indicator holder is designed to accommodate most sizes and shapes of bioindicators. Metal clips hold several common sizes of plastic tube bioindicators. Additionally, there is a cage (either made of composite plastic or copper) that can hold biological indicators of more uncommon sizes or those made of glass, which should not be held by spring clips. This cage is attached to the main body of the device by a threaded connection.

This device is unique for its ability to allow steam pass-through to autoclave bagged-waste as well as the ability to allow safe retrieval of a biological indicators placed near the center of a bag.

The overall length of device is 20 inches. The widest point of the device is 3 inches. This device is placed in a waste bag with the T-shaped handle protruding from the cinched opening. The composite end is placed center-mass and can hold several brands and types of biological indicators. This sheet also shows a detail of the internal spring snaps which allows extensions of tubing to be added or removed. Description of the reference characters: steam can enter the device at this location 1. Steam pass-through via hollow copper shaft allows proper permeation of the waste material in the autoclave bag; copper material for long autoclave life and possible antimicrobial properties 2. Composite collar allows autoclave bags to be cinched tight while still allowing steam in 3. Biological indicator holder is a composite material to prevent excessive heat transfer from the copper steam pass-through tube 4. Steam will enter the autoclave bag through this port 5. Springed snap buttons allow segments of the copper shaft to be added or removed based on the size of the autoclave bag 6.

This composite end is placed center-mass in a waste bag, and can hold various biological indicators. There are metal spring clips to hold plastic tube biological indicators. Additionally there is a cage that can hold more uncommon sizes of bioindicators or those made of glass. Description of the reference characters: Composite plastic material to minimize heat transfer from copper tubing to biological indicators 7. The end of the Composite biological indicator holder is threaded to attach a cage to hold uncommon sizes of bioindicators 8. Metal clips are interchangeable and multiple sizes. Can be attached to both sides via screws 9.

1. An autoclavable device comprising a steam pass-through tube, a composite collar around said tube, and a biological indicator holder at the end of said steam pass-through tube, whereby ensuring that an autoclave bag will receive proper steam permeation throughout the contents.

2. The device of claim 1 wherein the steam pass-through tube acts as a handle for the biological indicator holder whereby providing a method for safely inserting and later removing a biological indicator from the center of an autoclave bag.
